(19) United States
(12) Patent Application Publication
(10) Pub. No.: US 2026/0016454 A1
(43) Pub. Date: Jan. 15, 2026

(54) METHOD FOR EVALUATING CLIMATE INFLUENCING FACTORS OF VEGETATION PHOTOSYNTHESIS CONSIDERING TIME EFFECT

(71) Applicant: Fuzhou University, Minhou County (CN)

(72) Inventors: Juanzhu LIANG, Minhou County (CN); Xueyang HAN, Minhou County (CN)

(21) Appl. No.: 18/965,304

(22) Filed: Dec. 2, 2024

(30) Foreign Application Priority Data
Jul. 9, 2024 (CN) 202410914693.9

(51) Int. Cl. G01N 33/00

(52) U.S. Cl. CPC G01N 33/0098

(57) ABSTRACT

A method for evaluating climate influencing factors of vegetation photosynthesis considering a time effect includes obtaining solar-induced chlorophyll fluorescence (SIF) data and data of climatic factors and preprocessing the data. A climate variable is obtained according to a time effect of the preprocessed data. A partial correlation coefficient and a significance test value are obtained according to the climate variable. An optimal time effect obtained according to the partial correlation coefficient, and an optimal partial correlation coefficient is extracted according to the optimal time effect. Percentages of significant positive correlation and significant negative correlation in a total area under different time effects according to the partial correlation coefficient and the significance test value are obtained, and the percentages are compared to obtain a comparison result. An evaluation is then made according to the comparison result, an impact of climatic factors on vegetation photosynthesis after the time effect is considered.

### CROSS REFERENCE TO RELATED APPLICATION

This patent application claims the benefit and priority of Chinese Patent Application No. 2024109146939, filed with the China National Intellectual Property Administration on Jul. 9, 2024, the disclosure of which is incorporated by reference herein in its entirety as part of the present application.

### TECHNICAL FIELD

The present disclosure relates to the technical field of ecology and vegetation photosynthesis, and in particular, to a method for evaluating climate influencing factors of vegetation photosynthesis considering a time effect.

### BACKGROUND

In the context of increasingly severe global climate change, a terrestrial ecosystem, as one of the main carbon sinks of the earth's ecosystem, absorbs 30% or above of global carbon dioxide emissions and makes great contributions to mitigating climate change. As vegetation is used as a monitoring index reflecting an ecological environmental status under climate change, dynamic change thereof is a direct feedback of the environment to climate change. Therefore, it is a key policy to study driving factors of vegetation photosynthesis and fully tap the potential of the vegetation carbon sink to achieve global climate goals. Existing research shows that climate change has a significant impact on vegetation distribution, growing season, productivity, photosynthesis rate, and carbon absorption capacity.

However, in previous studies, the impact of multi-climatic factors on a time effect (lag effect and accumulation effect) of vegetation photosynthesis was often ignored, thus underestimating the impact of climatic factors on vegetation photosynthesis. Therefore, it is very necessary to design a method for evaluating climate influencing factors of vegetation photosynthesis considering a time effect.

### SUMMARY

An objective of the present disclosure is to provide a method for evaluating climate influencing factors of vegetation photosynthesis considering a time effect, to improve accuracy of evaluating an impact of climatic factors on vegetation photosynthesis.

To achieve the above objective, the present disclosure provides the following solutions.

A method for evaluating climate influencing factors of vegetation photosynthesis considering a time effect includes the following steps:
obtaining solar-induced chlorophyll fluorescence (SIF) data and data of multiple climatic factors, and preprocessing the data to obtain preprocessed data;
obtaining a climate variable according to a time effect of the preprocessed data;
obtaining a partial correlation coefficient and a significance test value according to the climate variable;
obtaining an optimal time effect according to the partial correlation coefficient, and extracting an optimal partial correlation coefficient according to the optimal time effect;
obtaining percentages of significant positive correlation and significant negative correlation in a total area under different time effects according to the partial correlation coefficient and the significance test value, and comparing the percentages to obtain a comparison result; and
evaluating, according to the comparison result, an impact of climatic factors on vegetation photosynthesis after the time effect is considered.

Optionally, the preprocessing the data includes projection conversion, outlier removal, resampling and image clipping.

Optionally, the obtaining a climate variable according to a time effect of the preprocessed data includes the following specific steps:
determining the time effect of the climatic factors, where the time effect includes no time effect, a lag effect, an accumulation effect, and combined lag-accumulation effects;
obtaining a time scale according to different time effects, where the time scale includes a lag scale, an accumulation scale and a lag-accumulation scale; and
obtaining climate variables of different time scales under different time effects according to the time effect and the time scale.

Optionally, a formula for calculating the climate variable is:

$${VPD}_{t ( m , n )} = \frac{1}{n} {\sum }_{i = 0}^{n} {VPD}_{t - m - i} ,$$

where VPD_{t(m,n)} is a climate variable of t months, m is a number of lagging months, n is a number of accumulation months, and VPDₜ₋ₘ₋ᵢ is a climate variable of a (m+i)^{th} month before the i^{th} month.

Optionally, a formula for calculating the partial correlation coefficient is:

$${r}_{xy . z} = \frac{{r}_{xy} - {r}_{xz} {r}_{yz}}{\sqrt{( 1 - {r}_{xz}^{2} ) ( 1 - {r}_{yz}^{2} )}} ,$$

where x is a first variable, y is a second variable, r_{xy.z} is a partial correlation coefficient between x and y, and z is a control variable; and r_{xy}, r_{xz} and r_{yz} are correlation coefficients between x and y, between x and z and between y and z, respectively.

Optionally, a formula for calculating the significance test value is:

$$t = r \sqrt{\frac{n - q - 2}{1 - {r}^{2}}} ,$$

where r is a partial correlation coefficient, n is a sample size, q is a partial correlation order, and t is the significance test value.

Optionally, the obtaining an optimal time effect according to the partial correlation coefficient, and extracting an optimal partial correlation coefficient according to the optimal time effect includes the following specific steps:
comparing partial correlation coefficients under different time effects, and taking a maximum number of lagging months and a maximum number of accumulation months in the partial correlation coefficients as an optimal lag time and an optimal accumulation time; and
extracting a partial correlation coefficient with a maximum absolute value according to the optimal lag time and the optimal accumulation time.

Optionally, when the significance test value is less than 0.05, a positive partial correlation coefficient indicates significant positive correlation, and a negative partial correlation coefficient indicates significant negative correlation.

According to the specific embodiment of the present disclosure, the present disclosure has the following technical effects: The present disclosure provides a method for evaluating climate influencing factors of vegetation photosynthesis considering a time effect. The method includes: obtaining solar-induced chlorophyll fluorescence (SIF) data and data of multiple climatic factors, and preprocessing the data to obtain preprocessed data; obtaining a climate variable according to a time effect of the preprocessed data; obtaining a partial correlation coefficient and a significance test value according to the climate variable; obtaining an optimal time effect according to the partial correlation coefficient, and extracting an optimal partial correlation coefficient according to the optimal time effect; obtaining percentages of significant positive correlation and significant negative correlation in a total area under different time effects according to the partial correlation coefficient and the significance test value, and comparing the percentages to obtain a comparison result; and evaluating, according to the comparison result, an impact of climatic factors on vegetation photosynthesis after the time effect is considered. According to the method, a partial correlation analysis method is used to evaluate the time effect of the climatic factors on vegetation photosynthesis, which improves accuracy of evaluating the impact of the climatic factors on vegetation photosynthesis.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a flowchart of a method for evaluating climate influencing factors of vegetation photosynthesis considering a time effect according to an embodiment of the present disclosure; and

FIG. 2 is a detailed flowchart of a method for evaluating climate influencing factors of vegetation photosynthesis considering a time effect according to an embodiment of the present disclosure.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the described embodiments are merely some rather than all of the embodiments of the present disclosure. All other embodiments obtained by those of ordinary skill in the art based on the embodiments of the present disclosure without creative efforts shall fall within the protection scope of the present disclosure.

In order to make the above objective, features and advantages of the present disclosure clearer and more comprehensible, the present disclosure is further described in detail below with reference to the accompanying drawings and specific implementations.

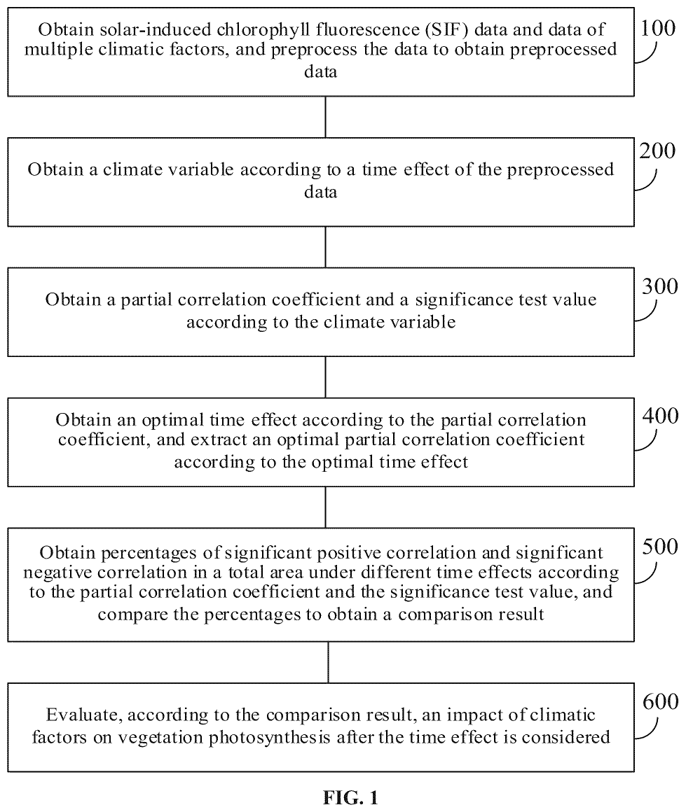

As shown in FIG. 1, an embodiment of the present disclosure provides a method for evaluating climate influencing factors of vegetation photosynthesis considering a time effect, including the following steps.

Step 100: Obtain SIF data and data of multiple climatic factors, and preprocess the data to obtain preprocessed data.

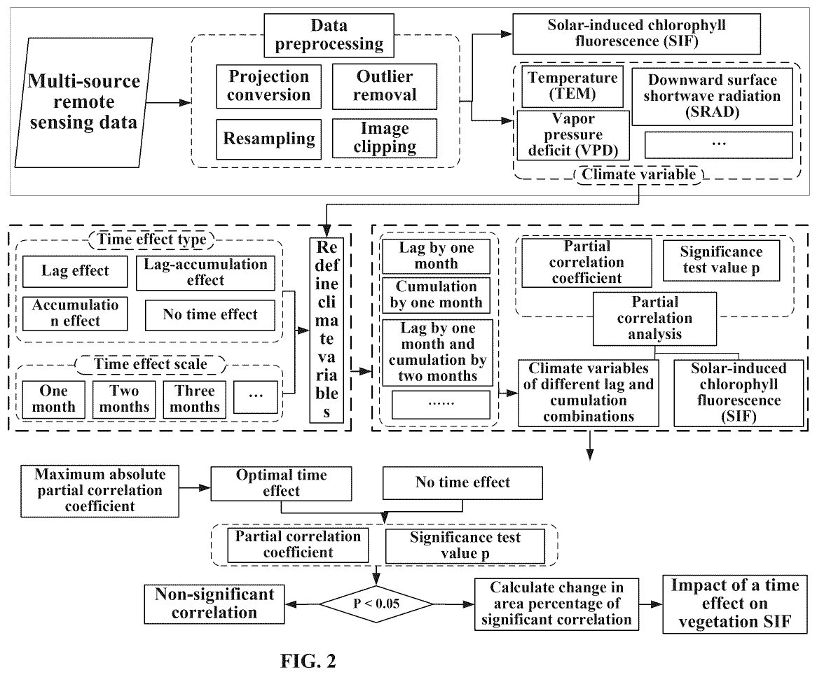

Specifically, as shown in FIG. 2, the preprocessing the data includes projection conversion, outlier removal, resampling and image clipping.

Step 200: Obtain a climate variable according to a time effect of the preprocessed data, including the following specific steps:
determining a time effect type of the preprocessed climatic factors on vegetation SIF, where the time effect type includes no time effect, a lag effect, an accumulation effect, and combined lag-accumulation effects;
obtaining a time scale according to different time effect types, such as lag by one month, cumulation by one month or lag by one month and cumulation by one month; and
obtaining climate variables of different time scales under different time effects according to the time effect type and the time scale.

Specifically, the climate variable of this embodiment takes a vapor pressure difference (VPD) as an example, and a formula for calculating the VPD is:

$${VPD}_{t ( m , n )} = \frac{1}{n} {\sum }_{i = 0}^{n} {VPD}_{t - m - i} ,$$

Where VPD_{t(m,n)} is a VPD of t months, m is a number of lagging months, n is a number of accumulation months, and VPDₜ₋ₘ₋ᵢ is a VPD of a (m+i)^{th} month before the i^{th} month.

Step 300: Obtain a partial correlation coefficient and a significance test value according to the climate variable.

Specifically, a formula for calculating the partial correlation coefficient is:

$${r}_{xy . z} = \frac{{r}_{xy} - {r}_{xz} {r}_{yz}}{\sqrt{( 1 - {r}_{xz}^{2} ) ( 1 - {r}_{yz}^{2} )}} ,$$

where x is a first variable, y is a second variable, r_{xy.z} is a partial correlation coefficient between x and y, and z is a control variable; r_{xy}, r_{xz} and r_{yz} are correlation coefficients between x and y, between x and z and between y and z, respectively, and x, y and z represent different climate variables respectively; and the partial correlation coefficient is between −1 and 1, and a greater absolute value thereof indicates stronger correlation.

Specifically, specific steps of obtaining the significance test value are as follows:
establishing a test hypothesis, assuming that there is no significant difference between the partial correlation coefficient and zero, that is, there is no correlation between the two variables; and
calculating the test statistic t, and determining a corresponding probability significance test value according to the value t. A formula for calculating the test statistic is:

$$t = r \sqrt{\frac{n - q - 2}{1 - {r}^{2}}} ,$$

where r is a partial correlation coefficient, n is a sample size, q is a partial correlation order, and t obeys a t distribution with a degree of freedom being n−q−2.

Step 400: Obtain an optimal time effect according to the partial correlation coefficient, and extracting an optimal partial correlation coefficient according to the optimal time effect, including the following specific steps:
comparing partial correlation coefficients of a pixel under different time effects, and taking m and n parameters with the maximum absolute partial correlation coefficients as an optimal lag time and an optimal accumulation time affecting SIF of the pixel, respectively, other pixels being in the same way; and
extracting a partial correlation coefficient of the pixel under the optimal time effect according to the optimal lag time and the optimal accumulation time, that is, a partial correlation coefficient with a maximum absolute value.

Step 500: Obtain percentages of significant positive correlation and significant negative correlation in a total area under different time effects according to the partial correlation coefficient and the significance test value, and compare the percentages to obtain a comparison result.

Specifically, a significance test value less than 0.05 indicates significant correlation, a positive partial correlation coefficient indicates significant positive correlation, and a negative partial correlation coefficient indicates significant negative correlation.

Specifically, different time effects are an optimal time effect and a neglected time effect.

Step 600: Evaluate, according to the comparison result, an impact of climatic factors on vegetation photosynthesis after the time effect is considered.

Corresponding to the above method, this embodiment further provides a system for evaluating climate influencing factors of vegetation photosynthesis considering a time effect. The system includes a data collection and preprocessing unit and a time lag and accumulation effect analysis unit of climatic factors on SIF.

The data collection and preprocessing unit is configured to obtain climate variable data in an SIF and TerraClimate data set of a region within 20 years (2002-2021), including multi-source remote sensing data such as temperature (TEM), precipitation (PPT), downward surface shortwave radiation (SRAD), soil moisture (SM) and vapor pressure deficit (VPD), and is further configured to preprocess image data.

The time lag and accumulation effect analysis unit of climatic factors on SIF is configured to redefine lag and accumulation climate variables, and calculate an optimal lag time and an optimal accumulation time corresponding to a partial correlation coefficient and a maximum absolute partial correlation coefficient between climate variables and SIF under different time lag and accumulation combinations, and is further configured to compare partial correlation coefficients between SIF and climatic factors under the optimal time effect and the neglected time effect, and quantitatively evaluate an impact of the time effect on vegetation SIF.

The present disclosure has the following beneficial effects:
1) By redefining of the lagging and accumulation climate variables and full consideration of a time impact of climate change on vegetation photosynthesis, the temporal and spatial evolution of regional photosynthesis is comprehensively monitored.
2) Multi-dimensional analysis and evaluation provide a variety of perspectives for the analysis of vegetation adaptation to climate change.
3) Accuracy of evaluating the impact of the climatic factors on vegetation photosynthesis is improved through partial correlation analysis and time effect analysis.

Embodiments of this specification are described in a progressive manner, and each embodiment focuses on the difference from other embodiments. For the same and similar parts between the embodiments, reference may be made to each other.

Specific examples are used for illustration of the principles and implementations of the present disclosure. The description of the above embodiments is merely used to help understand the method and its core ideas of the present disclosure. In addition, those of ordinary skill in the art can make modifications in terms of specific implementations and scope of use according to the ideas of the present disclosure. In conclusion, the content of this specification shall not be construed as limitations to the present disclosure.

What is claimed is:

1. A method for evaluating climate influencing factors of vegetation photosynthesis considering a time effect, comprising:
obtaining solar-induced chlorophyll fluorescence (SIF) data and data of multiple climatic factors, and preprocessing the data to obtain preprocessed data;
obtaining a climate variable according to a time effect of the preprocessed data;
obtaining a partial correlation coefficient and a significance test value according to the climate variable;
obtaining an optimal time effect according to the partial correlation coefficient, and extracting an optimal partial correlation coefficient according to the optimal time effect;
obtaining percentages of significant positive correlation and significant negative correlation in a total area under different time effects according to the partial correlation coefficient and the significance test value, and comparing the percentages to obtain a comparison result; and
evaluating, according to the comparison result, an impact of climatic factors on vegetation photosynthesis after the time effect is considered.

2. The method for evaluating climate influencing factors of vegetation photosynthesis considering a time effect according to claim 1, wherein the preprocessing the data comprises projection conversion, outlier removal, resampling and image clipping.

3. The method for evaluating climate influencing factors of vegetation photosynthesis considering a time effect according to claim 1, wherein the obtaining a climate variable according to a time effect of the preprocessed data comprises:
determining the time effect of the climatic factors, wherein the time effect comprises no time effect, a lag effect, an accumulation effect, and combined lag-accumulation effects;
obtaining a time scale according to different time effects, wherein the time scale comprises a lag scale, an accumulation scale, and a lag-accumulation scale; and
obtaining climate variables of different time scales under different time effects according to the time effect and the time scale.

4. The method for evaluating climate influencing factors of vegetation photosynthesis considering a time effect according to claim 3, wherein a formula for calculating the climate variable is:
$${VPD}_{t ( m , n )} = \frac{1}{n} {\sum }_{i = 0}^{n} {VPD}_{t - m - i} ,$$
wherein VPD_{t(m,n)} is a climate variable of t months, m is a number of lagging months, n is a number of accumulation months, and VPDₜ₋ₘ₋ᵢ is a climate variable of a (m+i)^{th} month before the i^{th} month.

5. The method for evaluating climate influencing factors of vegetation photosynthesis considering a time effect according to claim 1, wherein a formula for calculating the partial correlation coefficient is:
$${r}_{xy . z} = \frac{{r}_{xy} - {r}_{xz} {r}_{yz}}{\sqrt{( 1 - {r}_{xz}^{2} ) ( 1 - {r}_{yz}^{2} )}} ,$$
wherein x is a first variable, y is a second variable, r_{xy.z} is a partial correlation coefficient between x and y, and z is a control variable; and r_{xy}, r_{xz}, and r_{yz} are correlation coefficients between x and y, between x and z and between y and z, respectively.

6. The method for evaluating climate influencing factors of vegetation photosynthesis considering a time effect according to claim 1, wherein a formula for calculating the significance test value is:
$$t = r \sqrt{\frac{n - q - 2}{1 - {r}^{2}}} ,$$
wherein r is a partial correlation coefficient, n is a sample size, q is a partial correlation order, and t is the significance test value.

7. The method for evaluating climate influencing factors of vegetation photosynthesis considering a time effect according to claim 4, wherein the obtaining an optimal time effect according to the partial correlation coefficient, and extracting an optimal partial correlation coefficient according to the optimal time effect comprises:
comparing partial correlation coefficients under different time effects and taking a maximum number of lagging months and a maximum number of accumulation months in the partial correlation coefficients as an optimal lag time and an optimal accumulation time; and
extracting a partial correlation coefficient with a maximum absolute value according to the optimal lag time and the optimal accumulation time.

8. The method for evaluating climate influencing factors of vegetation photosynthesis considering a time effect according to claim 1, wherein when the significance test value is less than 0.05, a positive partial correlation coefficient indicates significant positive correlation, and a negative partial correlation coefficient indicates significant negative correlation.
